(19) United States
(12) Patent Application Publication
(10) Pub. No.: US 2008/0139450 A1
(43) Pub. Date: Jun. 12, 2008

(54) Antimicrobial Compositions and Uses Thereof

(76) Inventors: Srinivasa Madhyastha, Winnipeg (CA); Purushottam Gawande, Winnipeg (CA); Karen Lovetri, Winnipeg (CA)

Correspondence Address: MERCHANT & GOULD PC, P.O. BOX 2903, MINNEAPOLIS, MN, 55402-0903, US

(21) Appl. No.: 11/750,826

(22) Filed: May 18, 2007

Related U.S. Application Data
(63) Continuation-in-part of application No. 11/331,423, filed on Jan. 11, 2006.
(60) Provisional application No. 60/742,972, filed on Dec. 6, 2005.
(60) Provisional application No. 60/695,546, filed on Jul. 1, 2005.

(51) Int. Cl. A61K 31/155; A61P 31/04; A61K 38/02

(52) U.S. Cl. 514/2; 514/635

(57) ABSTRACT

The present invention includes compositions for preventing growth and proliferation of biofilm embedded microorganisms comprising: (a) a cationic polypeptide and (b) a bis-guanide or a salt thereof. The invention further provides methods for preparing objects with such compositions, and objects and consumables (such as household cleaners) comprising such compositions.

### CROSS REFERENCE TO RELATED APPLICATIONS

This application is a continuation-in-part of U.S. patent application Ser. No. 11/331,423, filed Jan. 11, 2006, and claims the benefit under 35 U.S.C. § 119(e) of U.S. Provisional Application No. 60/695,546, filed Jul. 1, 2005, and U.S. Provisional Application No. 60/742,972, filed Dec. 6, 2005, the entire disclosures of which are hereby incorporated by reference.

### FIELD OF THE INVENTION

The present invention relates to a novel antimicrobial composition that inhibits growth and proliferation of biofilm embedded microorganisms. A composition of the invention is useful in a variety of applications where inhibition of growth and proliferation of such microorganisms is desirable.

### BACKGROUND

Urinary tract infection (UTI) is the most common hospital-acquired infection, accounting for up to 40% of all nosocomial infections. The majority of cases of UTI are associated with the use of urinary catheters, including trans-urethral foley, suprapubic, and nephrostomy catheters. These urinary catheters are inserted in a variety of populations, including the elderly, stroke victims, spinal cord-injured patients, post-operative patients and those with obstructive uropathy. Despite adherence to sterile guidelines for the insertion and maintenance of urinary catheters, catheter-associated UTI continues to pose a major problem. For instance, it is estimated that almost one-quarter of hospitalized spinal cord-injured patients develop symptomatic UTI during their hospital course. Gram-negative bacilli account for almost 60-70%, Enterococci for about 25%, and Candida species for about 10% cases of catheter-associated UTI. Furthermore, indwelling medical devices including vascular catheters are becoming essential in the management of hospitalized patients by providing venous access. The benefit derived from these catheters as well as other types of medical devices such as peritoneal catheters, cardiovascular devices, orthopedic implants, and other prosthetic devices is often offset by infectious complications. The most common organisms causing these infectious complications are Staphylococcus epidermidis and Staphylococcus aureus. In the case of vascular catheters, these two organisms account for almost 70-80% of all infectious organisms, with Staphylococcus epidermidis being the most common organism. Candida albicans, a fungal agent, accounts for 10-15% of catheter infections.

In recent years, there have been numerous efforts to sequester antimicrobials and antibiotics on the surface of or within devices that are then placed in the vasculature or urinary tract as a means of reducing the incidence of device-related infections. These antimicrobial agents are of varying chemical composition and can include cationic polypeptides (protamine, polylysine, lysozyme, etc.), antiseptics (chlorhexidine, triclosan, etc.), surfactants (sodium dodecyl sulfate, Tween®-80, surfactin, etc.), quaternary ammonium compounds (benzalkonium chloride, tridodecyl methyl ammonium chloride, didecyl dimethyl ammonium chloride, etc.), silver ions/compounds, and nitrofurazone.

The main methods of antimicrobial catheter preparation include immersion or flushing, coating, drug-polymer conjugate and impregnating (Tunney et al., Rev. Med. Microbiol., 7(4):195-205, 1996). In a clinical setting, suitable catheters can be treated by immersion immediately prior to placement, which offers flexibility and control to clinicians in certain situations. Several studies have examined the clinical efficacy of catheters coated with antimicrobial agents. Polyurethane catheters coated with minocycline and EDTA showed potential in reducing recurrent vascular catheter-related infections (Raad et al., Clin. Infect. Dis., 25:149-151, 1997). Minocycline and rifampin coatings have been shown to significantly reduce the risk of catheter-associated infections (Raad et al., Crit. Care Med., 26:219-224, 1998). Minocycline coated onto urethral catheters has been shown to provide some protection against colonization (Darouicheet al., Int. J. Antimicrob. Ag., 8:243-247, 1997). Johnson et al. described substantial in vitro antimicrobial activity of a commercially available nitrofurazone coated silicone catheter (Antimicrob. Agents Chemother., 43:2990-2995, 1999). The antibacterial activity of silver-containing compounds as antimicrobial coatings for medical devices has been widely investigated. Silver-sulfadiazine used in combination with chlorhexidine has received particular interest as a central venous catheter coating (Stickler, Curr. Opin. Infect. Dis., 13:389-393, 2000; Darouiche et al., New Eng. J. Med., 340: 1-8, 1999).

The loading of antimicrobial agents into medical devices by immersion or coating technologies has the advantage of being relatively simple. However, the limited mass of drug that can be incorporated may be insufficient for a prolonged antimicrobial effect, and the release of the drug following clinical insertion of the device is rapid and relatively uncontrolled. A means of reducing these problems is by direct incorporation of the antimicrobial agent into the polymeric matrix of the medical device at the polymer synthesis stage or at the device manufacture stage. Rifampicin has been incorporated into silicone in an attempt to prevent infection of cerebrospinal fluid shunts with some success (Schierholz et al., Biomaterials, 18:839-844, 1997). Iodine has also been incorporated into medical device biomaterials. Coronary stents have been modified to have antithrombogenic and antibacterial activity by covalent attachment of heparin to silicone with subsequent entrapment of antibiotics in cross-linked collagen bound to the heparinized surface (Fallgren et al., Zentralbl. Bakteriol., 287:19-31, 1998).

Welle et al. disclosed the method of preparing a kit for flushing a medical device (U.S. Pat. No. 6,187,768). The kit includes a solution containing an antibiotic, an anticoagulant (protamine sulfate) and an antithrombotic agent or chelating agent useful for preventing infections caused by bacterial growth in catheters.

Raad et al. disclosed that pharmaceutical compositions of a mixture of minocycline and EDTA were useful in maintaining the patency of a catheter port (U.S. Pat. No. 5,362,754). Recently, Raad and Sheretz further disclosed that effective catheter flush solutions could be prepared with non-glycopeptide antimicrobial agent, an antithrombic agent, an anticoagulant, and a chelating agent selected from the group consisting of EDTA, EGTA and DTPA (U.S. Pat. No. 5,688,516).

Welle et al. teach the use of several anticoagulants for use in medical devices, including protamine sulfate (U.S. Pat. No. 6,187,768). Combinations of protamine sulfate and certain antibiotics have been shown to have synergistic effects on catheter-associated bacteria such as Pseudomonas aeruginosa and Staphylococcus epidermidis (Soboh et al., Antimicrob. Agents. Chemother., 39: 1281-1286, 1995; Richards et al., ASAIO Trans, 36:296-299). Kim et al. (Am. J. Kidney Dis., 39: 165-173, 2002) developed an antimicrobial-impregnated peritoneal dialysis catheter by impregnating the cuff and tubing with chlorhexidine, silver-sulfadiazine and triclosan in a polymer matrix. Fox et al. disclose medical devices having the synergistic composition comprising a silver salt and chlorhexidine (U.S. Pat. No. 5,019,096). Soloman et al. disclose anti-infective medical articles containing chlorhexidine (U.S. Pat. No. 6,261,271). Modak et al., in U.S. Pat. Nos. 6,706,024 and 6,843,784, disclose chlorhexidine, triclosan, and silver compound containing medical devices.

Antimicrobial compositions have found an increasing number of commercial and consumer uses, and an effective antimicrobial composition, such as a composition that inhibits growth and proliferation of biofilm embedded microorganisms is useful in a plethora of applications. Such an antimicrobial composition can either be used on its own, incorporated into a consumable, or incorporated into a surface desirable to be free of bacteria.

Antimicrobial compositions have been increasingly used in oral care, including incorporation of such compounds or compositions into toothpaste, mouth wash, chewing gum, breath mints, and similar consumables. Also for oral care, it is often desirable to have products such as dental floss, dentures and mouth guards with surfaces that are resistant to microbes.

Industrial applications to antimicrobial compounds include their use in dairy lines, either as a flush or wash for such lines, or incorporated within the lines, for example as a coating; liquid distribution lines in the food and beverage manufacturing or dispensing, for example, use as a coating in feeder lines for high sugar or syrup distribution in the manufacturing of soft drinks; pulp and paper mills (for biofouling); in the manufacturing and containment of cosmetics from production line equipment down to the end consumable, either incorporated within the cosmetic or coated on the jar containing the cosmetic; in water treatment facilities; in the leaching process used in mining; to prevent corrosion caused or accelerated by organisms, in oil and gas pipelines, in the souring of oil fields, and in cooling towers.

Consumer and light commercial uses of antimicrobial agents include their incorporation in general household disinfectants, laundry detergents, cleaning supplies, wound care, vacuum systems and vacuum filters, paint and wall coverings, humidifiers and humidifier filters, vacuum cleaners, toys, and incorporation into plastics for a variety of household items, including the inside and outside of washing machines, dishwashers, animal water dishes, bathroom tiles and fixtures, sealants and grout, towels, Tupperware, dishes, cutting boards, dish drying trays, bathtubs including whirlpool and jacuzzi bathtubs, fish ponds, swimming pools, bird baths, garden hose, planters and hot tubs.

Accordingly, a novel and effective antimicrobial composition, having a more potent antimicrobial effect or an antimicrobial effect at a lower concentration, is highly desirable. Such a composition is even more desirable if it is safe and non-harmful to humans or livestock. Such a composition is even more desirable when it is inexpensive to produce, or made from a synergistic or highly affective combination of products that are known and well characterized individually.

### SUMMARY OF THE INVENTION

An embodiment of the present invention provides a composition for preventing growth and proliferation of biofilm embedded microorganisms, said composition comprising: (a) a cationic polypeptide and (b) a bis-guanide or a salt thereof.

In an embodiment of the invention, a composition is useful for preventing growth and proliferation of biofilm embedded microorganisms on a device.

In an embodiment of the invention, a cationic polypeptide includes about 12.5 mg/ml and about 100 mg/ml of a composition.

In another embodiment of the invention, a bis-guanide is about 100 mg/ml and about 400 mg/ml of a composition.

In a further embodiment, a composition according to the invention is effective for preventing growth and proliferation of biofilm embedded bacteria.

Bacteria may include, but are not limited to, gram-negative bacteria such as Escherichia coli, Proteus mirabilis, Klebsiella pneumoniae, Pseudomonas aeruginosa, Klebsiella oxytoca, Providentia stuartii, Serratia marcescens, Porphyromonas gingivalis, Fusobacterium nucleatum, and Prevotella intermedia.

Bacteria may include, but are not limited to, gram-positive bacteria such as Enterococcus faecalis, Vancomycin Resistant Enterococci (VRE), Streptococcus viridans, Staphylococcus epidermidis, Staphylococcus aureus, Staphylococcus saprophyticus, Bacillus cereus, Streptococcus thermophilus, Clostridium perfringens, Listeria monocytogenes, Streptococcus mutans, Streptococcus sobrinus, Porphyromonas gingivalis and Actinomyces naeslundii.

In another embodiment, a composition is effective for preventing growth and proliferation of biofilm embedded fungi, which may include Candida albicans.

In a further embodiment, a cationic polypeptide is selected from the group consisting of protamine sulfate, defensin, lactoperoxidase, and lysozyme.

In a still further embodiment, the bis-guanide is selected from the group consisting of chlorhexidine, alexidine, and polymeric bis-guanides.

In a still further embodiment, a bis-guanide is a chlorhexidine base or a chlorhexidine salt.

A chlorhexidine salt may be selected from the group consisting of chlorhexidine diglucanate, chlorhexidine diacetate, and chlorhexidine dihydrochloride.

In a further embodiment, a cationic polypeptide is protamine sulfate and the bis-guanide is a chlorhexidine salt.

In a still further embodiment, a composition comprises about 100 mg/ml protamine sulfate and about 400 mg/ml chlorhexidine salt.

In yet a further embodiment, a composition according to the invention further comprises one or more ingredients such as water; a binding, bonding or coupling agent or cross-linking agent; a bis-phenol; a quaternary ammonium compound; a maleimide; an antibiotic; and a pH adjuster.

In another aspect, the present invention includes a method of preparing an object comprising treating at least one surface of the object with a composition according to the methods disclosed herein.

In an embodiment, an object is a device.

A further aspect provides a method of preparing an object comprising incorporating a composition according to the invention into polymers, which are used to form the object.

In an embodiment, an object is a device.

In another aspect, the present invention provides a method of preparing an object comprising coating a composition according to the invention onto at least one surface of the object.

In an embodiment, a composition comprises effective amounts of protamine sulfate and chlorhexidine salt.

In another embodiment, an object is a dairy line or a filter for a dairy line.

In another embodiment, an object is an apparatus or a processing line for manufacturing food or beverage.

In another embodiment, an object is an apparatus for cosmetic manufacturing.

In another embodiment, an object is a food, beverage, or cosmetic container.

In another embodiment, an object is a part of a water treatment facility or a cooling tower.

In another embodiment, an object is a heating, ventilating, and air conditioning (HVAC) system or a filter for an HVAC system.

In another embodiment, an object is a vacuum, a vacuum cleaner, or a vacuum or vacuum cleaner filter or bag.

In another embodiment, an object is an oil or gas pipeline.

In another embodiment, an object is a window, a door, or a window or door frame.

In another embodiment, an object is a humidifier or a humidifier filter.

In another embodiment, an object is a toy.

In another embodiment, an object is a component of a cooling tower.

In another embodiment, an object is a medical or dental instrument.

In another embodiment, an object is a household item, for example, a washing machine, a washing machine liner, a dishwasher, a dishwasher liner, an animal water dish, a bathroom tile, a bathroom fixture, a shower head, a sealant, grout, a towel, a food or beverage storage container, a dish, a cutting board, a dish drying tray, or a bathroom fixture such as a bath tub, a whirl pool bath tub, a sink, a bottle, a vacuum cleaner, a toilet lid, a toilet seat, a swimming pool liner, a swimming pool skimmer, a swimming pool filter, a hot tub line, a hot tub filter, a dish, a plate, a cup, a bowl, a fork, a knife, a spoon, a utensil, a hot tub, a counter top, or a toilet.

In another embodiment, an object is an outdoor water apparatus, such as a fish pond, a swimming pool, a bird bath, a garden hose, a planter, a hot tub, a water jug, a water sprinkling line, or a water sprinkler.

In another embodiment of the invention, a device is a medical device.

In another embodiment of the invention, a medical device may be a catheter.

A catheter may be an indwelling catheter such as a central venous catheter, a peripheral intravenous catheter, an arterial catheter, a hemodialysis catheter, an umbilical catheter, percutaneous nontunneled silicone catheter, a cuffed tunneled central venous catheter, or a subcutaneous central venous port.

A catheter may be an indwelling catheter such as urinary catheter, a peritoneal catheter, or a central venous catheter.

In another embodiment, a device includes catheters, pacemakers, prosthetic heart valves, prosthetic joints, voice prostheses, contact lenses, or intrauterine devices.

In a further aspect, the invention provides a composition for preventing infection, said composition comprising: (a) a cationic polypeptide and (b) a bis-guanide or a salt thereof.

In an embodiment, an infection is a device-related infection.

In another embodiment, a composition is incorporated into a consumable.

In another embodiment, a consumable is a toothbrush, toothpaste, mouth wash, dental floss, chewing gum, breath mint, denture, or mouth guard.

In another embodiment, a consumable is a general household disinfectant, a window cleaner, a bathroom cleaner, a kitchen cleaner, a floor cleaner, a fabric softener, laundry detergent, or a cleaning supply.

In another embodiment, a consumable is a bandage or adhesive bandage or wound dressing, for example, band aids, non-resorbable gauze/sponge dressing, hydrophilic wound dressing, occlusive wound dressing, hydrogel wound and burn dressing, spray-applicator, ointments, lotions, cream, and suture.

In another embodiment, a consumable is a cosmetic, such as a face powder, a lip balm, a lipstick, an eye liner, or a mascara.

In another embodiment, a consumable is a paint or a wall covering.

In another embodiment, a consumable is a humidifier filter.

In another embodiment, a consumable is a garbage bag.

In a further aspect, the invention provides a method of preparing a device comprising treating at least one surface of the device with (a) a cationic polypeptide and (b) a bis-guanide or a salt thereof.

In a further aspect, the invention provides a composition comprising (a) a cationic polypeptide, (b) a bis-guanide or a salt thereof, and (c) a medical device on which said cationic polypeptide and said bis-guanidine or salt thereof is coated, incorporated, or treated.

In a further aspect, the invention provides a use of any of the compositions described herein for prevention and treatment of infections in humans and animals.

In a further aspect, the invention provides a use of any of the compositions described herein in the preparation of a medical device for implantation in a mammal.

### BRIEF DESCRIPTION OF THE FIGURES

FIG. 1 is a bar graph illustrating the effect of a negative control (NC) (solution without an active ingredient), 50 μg/ml protamine sulfate (PS), 12.5 μg/ml chlorhexidine salt (CHX), and a combination of 50 μg/ml protamine sulfate and 12.5 μg/ml chlorhexidine salt (PS+CHX) on the number (CFU) of biofilm embedded E. coli.

FIG. 2 is a bar graph illustrating the effect of a negative control (NC) (solution without an active ingredient), 25 μg/ml protamine sulfate (PS), 25 μg/ml chlorhexidine salt (CHX), and a combination of 25 μg/ml protamine sulfate and 25 μg/ml chlorhexidine salt (PS+CHX) on the number (CFU) of biofilm embedded Pseudomonas aeruginosa.

FIG. 3 is a bar graph illustrating the enhanced effect of a negative control (NC) (solution without an active ingredient), 12.5 μg/ml protamine sulfate (PS), 12.5 μg/ml chlorhexidine salt (CHX), and a combination of 12.5 μg/ml protamine sulfate and 12.5 μg/ml chlorhexidine salt (PS+CHX) on the number (CFU) of biofilm embedded Staphylococcus epidermidis.

FIG. 4 is a bar graph illustrating the anti-adherence effects of silicone catheters coated with 100 mg/ml protamine sulfate (PS), 100 mg/ml chlorhexidine salt (CHX), and a combination of 100 mg/ml protamine sulfate and 100 mg/ml chlorhexidine salt (PS+CHX) on E. coli.

FIG. 5 is a bar graph illustrating the enhanced anti-adherence effect of silicone catheters coated with 100 mg/ml protamine sulfate (PS), 100 mg ml chlorhexidine salt (CHX), and a combination of 100 mg/ml protamine sulfate and 100 mg ml chlorhexidine salt (PS+CHX) on Pseudomonas aeruginosa.

FIG. 6 is a bar graph illustrating the anti-adherence effect of the silicone catheters coated with 100 mg/ml protamine sulfate (PS), 100 mg/ml chlorhexidine salt (CHX), and a combination of 100 mg/ml protamine sulfate and 100 mg/ml chlorhexidine salt (PS+CHX) on Staphylococcus epidermidis.

FIG. 7 is a line graph illustrating the durability of anti-adherence activity of 100 mg/ml protamine sulfate (PS) and 400 mg/ml chlorhexidine salt (CHX) coated silicone catheter against E. coli.

FIG. 8 is a line graph illustrating the durability of anti-adherence activity of 100 mg/ml protamine sulfate (PS) and 400 mg/ml chlorhexidine salt (CHX) coated silicone catheters against Staphylococcus epidermidis.

FIG. 9 is a bar graph illustrating the effect of a negative control (NC) (solution without an active ingredient), 0.4 μg/ml protamine sulfate (PS), 0.4 μg/ml chlorhexidine (CHX), and a combination of 0.4 μg/ml protamine sulfate and 0.4 μg/ml chlorhexidine (PS+CHX) on the number (CFU) of biofilm embedded Escherichia coli.

FIG. 10 a bar graph illustrating the effect of a negative control (NC) (solution without an active ingredient), 6.25 μg/ml protamine sulfate (PS), 3 μg/ml chlorhexidine (CHX), and a combination of 6.25 μg/ml protamine sulfate and 3 μg/ml chlorhexidine (PS+CHX) on the number (CFU) of biofilm embedded Bacillus cereus.

### DETAILED DESCRIPTION

Compositions comprising at least one cationic polypetide and at least one bis-guanide have enhanced antimicrobial activity. In particular, such compounds are effective for preventing growth and proliferation of microorganisms, including both bacterial and fungal species, embedded in biofilms. An enhanced antimicrobial activity is evidenced by the small quantities of each of these compounds that need to be used to produce an effective antimicrobial composition. A necessary overall amount of the compounds is less than that which would be required if any of the compounds were to be used on their own. In particular. it is possible to use small amounts of a cationic polypeptide, which is biologically acceptable, and a small amount of bis-guanide, which is biologically acceptable at lower concentrations and are effective antimicrobials.

Accordingly, an embodiment of the present invention provides compositions for preventing growth and proliferation of biofilm embedded microorganisms comprising: (a) a cationic polypeptide and (b) a bis-guanide or salt thereof.

An embodiment of the present invention also provides compositions for preventing infection caused or exacerbated by implanted medical devices or catheters, such as urinary tract infections caused by indwelling catheters, by coating said medical devices or catheters with said composition, such composition comprising (a) a cationic polypeptide and (b) a bis-guanide or salt thereof.

A synergistic antimicrobial composition of the invention requires remarkably small amounts of active ingredients (compared to that which has been used in the past) to be effective. A composition according to the invention may have properties that include those of separate compounds but go beyond them in efficacy and scope of application. Extremely low levels, and hence increased efficacy, of active compounds or ingredients, make embodiments of this invention very desirable and relatively economical to manufacture, although higher concentrations of these compounds can be used if it is desired for certain applications. A further advantage of using these compositions is the effectiveness for preventing growth of biofilm embedded bacteria and fungus, and in particular, bacterial and fungal species that colonize medical devices such as catheters. Examples of cationic polypeptides useful for preparing compositions of the invention include, but are not limited to, protamine sulfate, defensin, lactoperoxidase, and lysozyme. In a preferred embodiment of the invention, the cationic polypeptide is protamine sulfate.

An amount of cationic polypeptide included in the composition is preferably about 10 mg/ml to about 200 mg/ml and more preferably about 12.5 mg/ml to about 100 mg/ml. The higher end of this range can be used to prepare a concentrated product which may be diluted prior to use.

Examples of bis-guanides useful for preparing the compositions of the invention include, but are not limited to chlorhexidine, alexidine, or polymeric bis-guanides. A bis-guanide may be in the form of a suitable salt. Bis-guanide salts are well known. In a preferred embodiment of the invention, compositions are prepared using a chlorhexidine salt, and more preferably of chlorhexidine diglucanate, chlorhexidine diacetate, or chlorhexidine dihydrochloride.

An amount of bis-guanide included in a composition is preferably about 10 mg/ml to about 400 mg/ml and more preferably about 100 mg/ml to about 400 mg/ml. The higher end of this range can be used to prepare a concentrated product that may be diluted prior to use.

Higher concentrations of a compound can be used for certain applications depending on targeted bacteria and a device to be treated. Suitable working concentrations can easily be determined using known methods.

In a preferred embodiment of the invention, a composition comprises protamine sulfate as the cationic polypeptide and a chlorhexidine salt as the bis-guanide. In a further preferred embodiment, the composition includes about 100 mg/ml of protamine sulfate and about 100 mg/ml of a chlorhexidine base or salt.

Compositions of the invention can be prepared using known methods. Generally, components are dissolved in a suitable solvent, such as water, glycerol, organic acids, or other suitable solvents

Compositions of the invention may include any number of well known active components and base materials. Compositions may further comprise ingredients such as, but not limited to: suitable solvents such as water; antimicrobials such as antibacterials and antifungals; a binding, bonding, coupling agent, cross-linking agent; or a pH adjuster.

Compositions of the invention may further comprise additional antimicrobial ingredients such as bis-phenols, N-substituted maleimides, and quaternary ammonium compounds. Examples of bis-phenols useful for preparing compositions of the present invention include, but are not limited to, triclosan and hexachlorophene. Examples of N-maleimides useful for preparing compositions of the present invention include, but are not limited: to N-ethylmaleimide (NEM), N-phenylmaleimide (PheM), N-(1-pyrenyl)maleimide (PyrM), naphthalene-1,5-dimaleimide (NDM), N,N′-(1,2-phenylene)dimaleimide (oPDM), N,N′-1,4-phenylene dimaleimide (pPDM), N,N′-1,3-phenylene dimaleimide (mPDM), and 1,1-(methylenedi-4,1-phenylene)bismaleimide (BM). Examples of quaternary ammonium compounds useful for preparing compositions of the present invention include, but are not limited to benzalkonium chloride, tridodecyl methyl ammonium chloride, and didecyl dimethyl ammonium chloride.

Other possible components of the composition include, but are not limited to, buffer solutions, phosphate buffered saline, saline, polyvinyl, polyethylene, polyurethane, polypropylene, silicone (e.g., silicone lassoers and silicone adhesives), polycarboxylic acids, (e.g., polyacrylic acid, polymethacrylic acid, polymaleic acid, poly-(maleic acid monoester), polyaspartic acid, polyglutamic acid, aginic acid or pectimic acid), polycarboxylic acid anhydrides (e.g., polymaleic anhydride, polymethacrylic anhydride or polyacrylic acid anhydride), polyamines, polyamine ions (e.g., polyethylene imine, polyvinylamine, polylysine, poly-(dialkylaminoethyl methacrylate), poly-(dialkylaminomethyl styrene) or poly-(vinylpyridine), polyammonium ions (e.g., poly-(2-methacryloxyethyl trialkyl ammonium ion), poly-(vinylbenzyl trialkyl ammonium ions), poly-(N,N-alkylypyridinium ion) or poly-(dialkyloctamethylene ammonium ion) and polysulfonates (e.g. poly-(vinyl sulfonate) or poly-(styrene sulfonate), collodion, nylon, rubber, plastic, polyesters, Dacron® (polyethylene teraphthalate), Teflon® (polytetrafluoroethylene), latex, and derivatives thereof, elastomers and Dacron® (sealed with gelatin, collagen or albumin), cyanoacrylates, methacrylates, papers with porous barrier films, adhesives, e.g., hot melt adhesives, solvent based adhesives, and adhesive hydrogels, fabrics, and crosslinked and non-crosslinked hydrogels, and any other polymeric materials which facilitate dispersion of the active components and adhesion of the biofilm penetrating coating to at least one surface of the medical device. Linear copolymers, cross-linked copolymers, graft polymers, and block polymers, containing monomers as constituents of the above-exemplified polymers may also be used.

Examples of biofilm embedded bacteria that may be inhibited using compositions according to the invention include gram-negative bacteria such as, but not limited to: Escherichia coli, Proteus mirabilis, Klebsiella pneumoniae, Pseudomonas aeruginosa, Klebsiella oxytoca, Providentia stuartii, Serratia marcescens, Fusobacterium nucleatum, Porphyromonas gingivalis and Prevotella intermedia, and gram-positive bacteria such as, but not limited to: Enterococcus faecalis, Vancomycin Resistant Enterococci (VRE), Streptococcus viridans, Staphylococcus epidermidis, Staphylococcus aureus or Staphylococcus saprophyticuis, Bacillus cereus, Streptococcus thermophilus, Clostridium perfringens, Listeria monocytogenes, Streptococcus mutans, Streptococcus sobrinus and Actinomyces naeslundii. These bacteria are commonly found associated with medical devices including catheters.

Compositions according to the invention can also be used to inhibit growth and proliferation of biofilm embedded fungi such as Candida albicans, Candida parapsilosis, and Candida utilis. In another aspect, the present invention provides a method of preparing an object, such as a device comprising treating at least one surface of the object with a cationic polypeptide and bis-guanide composition according to the invention. In a preferred embodiment of the invention, a composition used to prepare a device comprises an effective amount of protamine sulfate as the cationic polypeptide and a chlorhexidine salt as the bis-guanide.

An object may be any object which is desirable to be microorganism resistant, such as a home product, an industrial product, a medical product or medical device, a piece of apparel or a textile, a building product, etc.

In another aspect, the present invention provides a composition suitable for coating an object which is desirable to be microorganism resistant, for example a paint, wall covering, or protective plastic coating.

The term “effective” refers to a sufficient amount of active components to substantially prevent growth or proliferation of biofilm embedded microorganisms on at least one surface of a medical device coated with an embodied composition; and as a sufficient amount of the active components to substantially penetrate, or break-up, a biofilm on at least one surface of a medical device, thereby facilitating access of active components, antimicrobial agents, and/or antifungal agents to microorganisms embedded in a biofilm, and thus, removal of substantially all microorganisms from at least one surface of a medical device treated with a solution of an embodied composition. An amount will vary for each active component and upon known factors such as pharmaceutical characteristics, type of medical device, degree of biofilm embedded microorganism contamination, use, and length of use.

Examples of devices that can be treated using compositions of the invention include medical devices such as tubing and other medical devices, such as catheters, pacemakers, prosthetic heart valves, prosthetic joints, voice prostheses, contact lenses, and intrauterine devices.

Medical devices include disposable or permanent or indwelling catheters, (e.g., central venous catheters, dialysis catheters, long-term tunneled central venous catheters, short-term central venous catheters, peripherally inserted central catheters, peripheral venous catheters, pulmonary artery Swan-Ganz catheters, urinary catheters, and peritoneal catheters), long-term urinary devices, tissue bonding urinary devices, vascular grafts, vascular catheter ports, wound drain tubes, ventricular catheters, hydrocephalus shunts, heart valves, heart assist devices (e.g., left ventricular assist devices), pacemaker capsules, incontinence devices, penile implants, small or temporary joint replacements, urinary dilator, cannulas, elastomers, hydrogels, surgical instruments, dental instruments, tubings, such as intravenous tubes, breathing tubes, dental water lines, dental drain tubes, and feeding tubes, fabrics, paper, indicator strips (e.g., paper indicator strips or plastic indicator strips), adhesives (e.g., hydrogel adhesives, hot-melt adhesives, or solvent-based adhesives), bandages, orthopedic implants, and any other device used in the medical field.

Medical devices also include any device which may be inserted or implanted into a human being or other animal, or placed at the insertion or implantation site such as the skin near the insertion or implantation site, and which include at least one surface which is susceptible to colonization by biofilm embedded microorganisms.

Medical devices include surfaces of equipment in operating rooms, emergency rooms, hospital rooms, clinics, and bathrooms.

Implantable medical devices include orthopedic implants, which may be inspected for contamination or infection by biofilm embedded microorganisms using endoscopy. Insertable medical devices include catheters and shunts, which can be inspected without invasive techniques such as endoscopy.

Medical devices may be formed of any suitable metallic materials or non-metallic materials. Examples of metallic materials include, but are not limited to, titanium, and stainless steel, and derivatives or combinations thereof. Examples of non-metallic materials include, but are not limited to, thermoplastic or polymeric materials such as rubber, plastic, polyesters, polyethylene, polyurethane, silicone, Gore-Tex® (polytetrafluoroethylene), Dacron® (polyethylene tetraphthalate), Teflon® (polytetrafluoroethylene), latex, elastomers, and Dacron® sealed with gelatin, collagen, or albumin, and derivatives or combinations thereof.

Examples of other objects that can be treated or coated using compositions of the invention, or in which compositions of the invention can be incorporated, include toothpaste, mouthwash, dental floss, chewing gum, breath mint, dentures, mouth guards, dairy lines, apparatus for pulp and paper mills, apparatus used in food and beverage manufacturing or distribution industry, such as syrup or water lines, general household disinfectant, laundry detergent, cleaning supplies, fruit and vegetable wash, adhesive bandages, bandages, wound dressings, ointments, lotions, cosmetics, cosmetic containers, equipment for water treatment facilities, equipment involved in the leaching process in mining, HVAC (Heating, Ventilation and Air Conditioning) systems and filters thereto, vacuums, vacuum cleaners and vacuum and vacuum cleaning bags and filters, pipelines for oil and gas, paint and wall coverings, windows, doors and window and door frames, humidifier and humidifier filters, toys, including plastic toys, equipment used in cooling towers, medical and dental instruments, incorporating or coating of plastics for a variety of household items, such as washing machine and washing machine liners, dishwasher and dishwasher liners, animal water dishes, bathroom towels and fixtures, sealants and grout, towels, food and beverage storage containers including tupperware, dishes, cutting boards, dish drying trays, whirlpool bath tubs, toilets and toilet seats, other acrylic bath tubs, sinks, taps and water spouts, outdoor pond liners, swimming pool, swimming pool liners, swimming pool equipment and filters, bird baths, garden hoses, planters, hot tubs, garbage bags, etc.

In a preferred embodiment, a method of treating at least one surface of an object such as a medical device comprises contacting the object with a composition according to the invention. As used herein, the term “contacting” includes, but is not limited to: coating, spraying, soaking, rinsing, flushing, submerging, and washing. An object to be coated is contacted with a composition for a period of time sufficient to remove substantially all biofilm embedded microorganisms from a treated surface of the object.

In a more preferred embodiment, an object, such as a medical device, is submerged in a composition for at least 5 minutes. Alternatively, an object may be flushed with a composition. For an object such as tubing, (e.g., a dental unit waterline or a dairy line or a food and beverage processing line), a composition may be poured into the tubing while both ends of the tubing are clamped such that the composition is retained within the lumen of the tubing. The tubing is then allowed to remain filled with the composition for a period of time sufficient to remove substantially all of the microorganisms from at least one surface. Generally, rubbing can be filled for at least about 1 minute to about 48 hours. Alternatively, tubing may be flushed by pouring a composition into the lumen of the tubing for an amount of time sufficient to prevent substantial growth of all biofilm embedded microorganisms. Such flushing may be required only once, or may be required at regular intervals over the lifetime of use of the tubing. Concentrations of active components in a composition may vary as desired or necessary to decrease the amount of time a composition is in contact with a medical device.

In another embodiment, a method for treating a surface of an object, a composition of the invention may also include an organic solvent, a material penetrating agent, or adding an alkalinizing agent to the composition, to enhance reactivity of a surface of the object with the composition. An organic solvent, material penetrating agent, and/or alkalinizing agent are those which preferably facilitate adhesion of a composition to at least one surface of the object.

Another aspect provides a method of coating a composition of the invention onto at least one surface of an object. In an embodiment, an object is a device such as a medical device. Broadly, a method for coating a medical device includes steps of providing a medical device; providing or forming a composition coating; and applying a composition coating to at least one surface of the medical device in an amount sufficient to substantially prevent growth or proliferation of biofilm embedded microorganisms on at least one surface of the medical device. In a specific embodiment, a method for coating a medical device includes steps of forming a composition of the invention of an effective concentration for activating an active component, thereby substantially preventing growth or proliferation of microorganisms on at least one surface of the medical device, wherein the composition of the invention is formed by combining an active component and a base material. At least one surface of a medical device is then contacted with a composition of the invention under conditions wherein the composition of the invention covers at least one surface of the medical device. The term “contacting” further includes, but is not limited to: impregnating, compounding, mixing, integrating, coating, spraying and dipping. This description, which is taught for a medical device, could easily and readily be used for many other objects for which it is desirable to have an antimicrobial coating.

In another embodiment of a method for coating an object, a composition coating is formed by combining an active component and a base material at room temperature and mixing a composition for a time sufficient to evenly disperse active agents in the composition prior to applying the composition to a surface of the device. An object may be contacted with a composition for a period of time sufficient for a composition to adhere to at least one surface of the device. After a composition is applied to a surface of an object, it is allowed to dry.

An object is preferably placed in contact with a composition by dipping the object in the composition for a period of time from about 30 seconds to about 180 minutes at a temperature ranging from about 25° C. to about 60° C. Preferably, an object is placed in contact with a composition by dipping the object in the composition for about 60 minutes at a temperature of about 37° C. The object is removed firm a composition and then allowed to dry. An object device may be placed in an oven or other heated environment for a period of time sufficient for a composition to dry.

Although one layer, or coating, of a composition is believed to provide a desired composition coating, multiple layers can be used. Multiple layers of a composition can be applied to at least one surface of an object by repeating steps described above. Preferably, an object is contacted with a composition three times, allowing the composition to dry on at least one surface of the object prior to contacting the object with the composition for each subsequent layer. Thus, an object preferably includes three coats, or layers, of a composition on at least one surface of the object.

In another embodiment, a method for coating an object such as a medical device with a composition coating includes steps of forming a composition coating of an effective concentration to substantially prevent the growth or proliferation of biofilm embedded microorganisms on at least one surface of an object by dissolving an active component in an organic solvent, combining a material penetrating agent to the active component(s) and organic solvent, and combining an alkalinizing agent to improve reactivity of the material of the object. A composition is then heated to a temperature ranging from about 30° C. to about 60° C. to enhance adherence of a composition coating to at least one surface of the device. A composition coating is applied to at least one surface of the object, preferably by contacting the composition coating to the at least one surface of the object for a sufficient period of time for the composition coating to adhere to at least one surface of the object. The object is then removed from a composition coating and allowed to dry, preferably, for at least 18 hours at room temperature. The object may then be rinsed with a liquid, such as water and allowed to dry for at least 2 hours, and preferably 4 hours, before being sterilized. To facilitate drying of a composition of the invention onto a surface of the object, the object may be placed into a heated environment such as an oven.

In another aspect, the invention provides a method of incorporating a composition according to the invention into an object such as a medical device. An object can be a medical device where a composition is incorporated into a material forming the medical device during formation of the medical device. For example, a composition may be combined with a material forming the medical device, e.g., silicone, polyurethane, polyethylene, Gore-Tex® (polytetrafluoroethylene), Dacron® (polyethylene tetraphthalate), and Teflon® (polytetrafluoroethylene), and/or polypropylene, and extruded with the material forming the medical device, thereby incorporating the composition into material forming the medical device. In this embodiment, the composition may be incorporated in a septum or adhesive, which is placed at the medical device insertion or implantation site. One example of a medical device having a composition incorporated into the material forming the medical device in accordance with this embodiment is a catheter insertion seal having an adhesive layer described below in greater detail. Another example of a medical device having a composition incorporated into the material is an adhesive. A composition of the invention can be integrated into an adhesive, such as tape, thereby providing an adhesive, which may prevent growth or proliferation of biofilm embedded microorganisms on at least one surface of the adhesive.

Although the invention has been described with reference to illustrative embodiments, it is understood that the invention is not limited to these precise embodiments and that various changes and modifications may be effected therein by one skilled in the art. All changes and modifications are intended to be encompassed in the appended claims.

### EXAMPLES

### Example 1

### Enhanced Effect of a Protamine Sulfate (PS) and Chlorhexidine Salt (CHX) Combination on Biofilm Embedded Catheter-Associated Bacteria

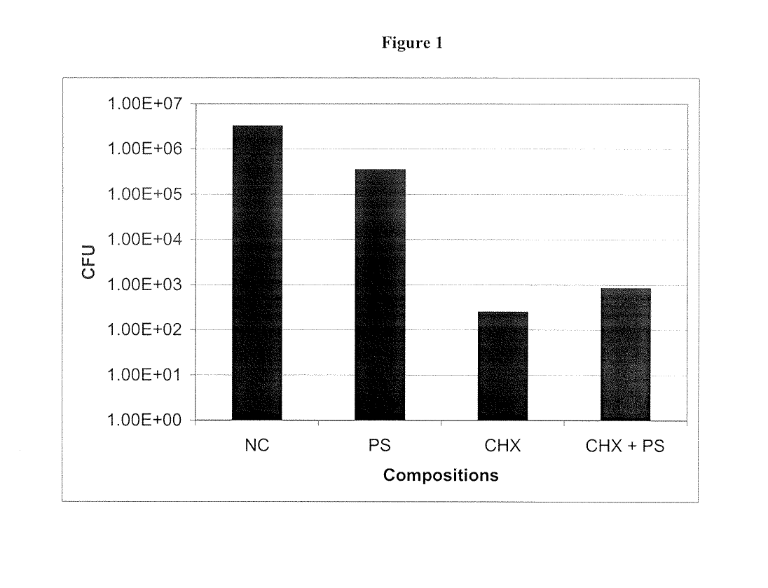

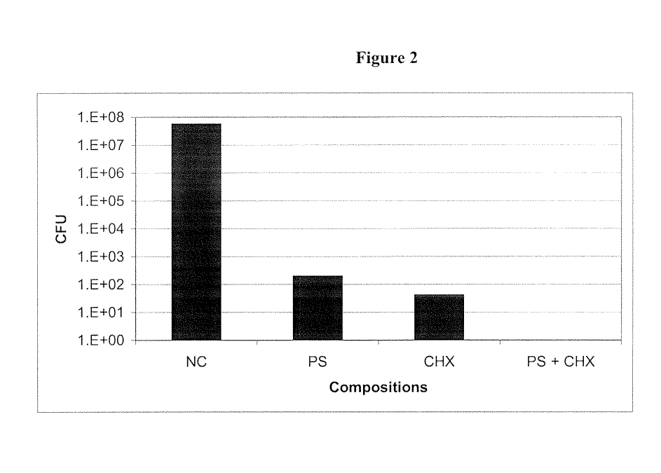

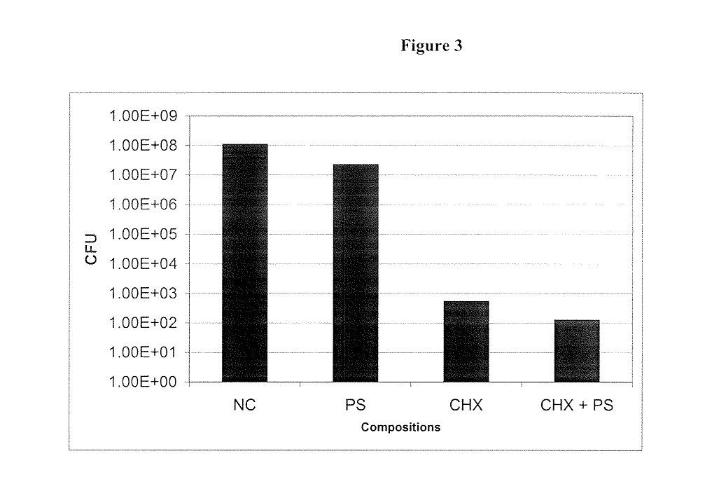

In vitro microplate assays were performed to determine the enhanced effects of protamine sulfate and chlorhexidine salt combination on the growth of biofilm embedded biofilm forming catheter-associated bacteria such as E. coli, Pseudomonas aeruginosa and Staphylococcus epidermidis. Overnight culture of each bacterial strain grown in Luria-Bertani (LB) or Tryptic Soy Broth (TSB) was used as inoculum. Bacteria were grown in Colony Forming Antigen (CFA) medium (for gram-negative) or in TSB (for gram-positive) on a 12-well microplate in the absence and presence of each test compound (PS or CHX) separately and together (PS+CHX) at 12.5, 25, or 50 μg/ml. The plate was incubated at 37° C. for 24 hours. Media containing planktonic cells in each well were removed gently and rinsed with sterile water. A known volume of water was added to each well and sonicated for 30 seconds. The transfer of contents of each well into a sterile tube and vortexing for a minute was followed by 10-fold serial dilution and plating on agar plates using a spreader. After incubating the plates at 37° C. for 24 hours, the colonies forming units (CFU) were counted. Although chlorhexidine salt was more effective than protamine sulfate in inhibiting the growth of all three biofilm embedded test organisms, the combination of protamine sulfate and chlorhexidine salt had an enhanced inhibitory effect on Pseudomonas aeruginosa and S. epidermidis (FIGS. 1-3).

### Example 2

### Inhibitory Activity of Protamine Sulfate (PS) and Chlorhexidine Salt ((CHX) Combination-Coated Silicone Catheter Against Catheter-Associated Bacteria

The antimicrobial activity of PS+CHX coated and uncoated 1 cm silicone catheter sections were assessed using Kirby-Bauer technique as previously described by Sheretz et al. (Antimicrob. Agents. Chemother., 33: 1174-1178, 1989). The catheters were coated by dipping in PS (100 mg/ml)+CHX (400 mg/ml) solution followed by drying as described in U.S. Pat. No. 6,475,434. The catheters were gas-sterilized with ethylene oxide. Catheter-associated microorganisms such as E. coli, Proteus mirabilis, Pseudomonas aeruginosa, Klebsiella pneumoniae, Enterococcus faecalis, Vancomycin Resistant Enterococci (VRE), Staphylococcus epidermidis, Staphylococcus aureus and Candida albicans were grown in nutrient broth for 18 hours at 37° C. An appropriate inoculum of each bacterial or yeast strain was used to prepare spread plates. The coated and uncoated catheter sections were then carefully pressed into the center of each of the plates. Following incubation for 24 hours at 37° C., the zones of inhibition surrounding each of the sections were measured at the aspects of perpendicular to the long axes. The zone of inhibition varied from organism to organism ranging from 6 mm to 21 mm (Table 1). The coated catheter had a significant inhibitory activity against E. coli, Staphylococcus epidermidis, Staphylococcus aureus, and Candida albicans.

TABLE 1
Inhibitory activity of the protamine sulfate (PS) + chlorhexidine
salt (CHX)-coated silicone catheter against catheter-associated
microorganisms

| Organism | Inhibition Zone (mm) |
|---|---|
| E. coli | 14 ± 4.2 |
| Proteus mirabilis | 8 ± 0 |
| Pseudomonas aeruginosa | 6 ± 0 |
| Klebsiella pneumoniae | 10 ± 2.8 |
| Enterococcus faecalis | 13 ± 1.4 |
| Vancomycin Resistant Enterococci (VRE) | 13 ± 1.4 |
| Staphylococcus epidermidis | 19 ± 0 |
| Staphylococcus aureus | 21 ± 4.2 |
| Candida albicans | 16.5 ± 3.5 |

### Example 3

### Anti-Adherence Effect of Protamine Sulfate (PS) and Chlorhexidine Salt (CHX) Combination-Coated Silicone Catheter on Catheter-Associated Bacteria

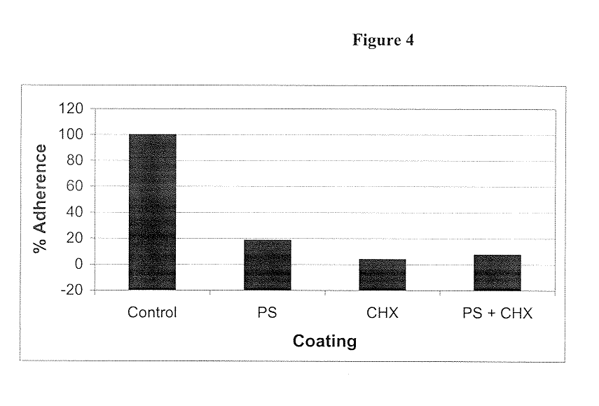

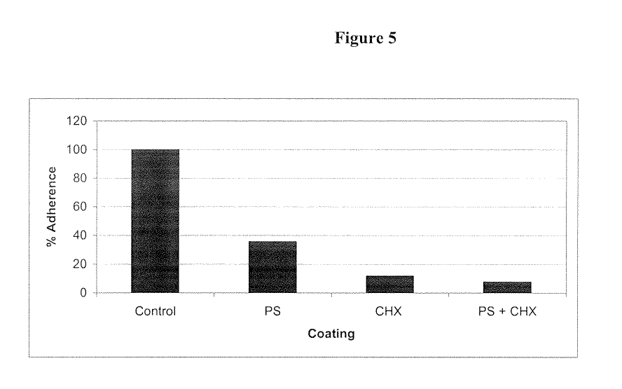

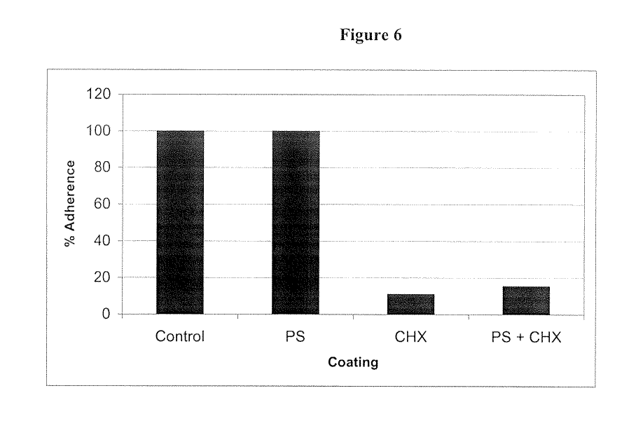

The ability of PS+CHX, PS, and CHX coated silicone catheters to resist bacterial colonization was tested by exposing uncoated and coated sections to E. coli, Pseudomonas aeruginosa, and Staphylococcus epidermidis in triplicate. The silicone catheters were coated with PS (100 mg/ml), CHX (100 mg/ml) and PS (100 mg/ml)+CHX (100 mg/ml), and gas-sterilized with ethylene oxide. The coated catheter sections were incubated in sterile artificial urine at 37° C. for 24 hours at 100 rpm prior to challenging with the bacteria. Following the incubation, the catheter sections were rinsed with sterile water and incubated in a bacterial culture in BHI medium at 37° C. for 3 hours at 100 rpm. After 3 hours of incubation, the sections were washed twice gently. Each washed section was transferred into a sterile tube containing 1 ml sterile water and subjected to sonication for 30 seconds followed by 1 minute vortexing. Further, each section was serially diluted using sterile water and plated on LB agar. The plates were incubated for 24 hours at 37° C. and the colonies (CFU) were counted. The CHX alone-coated catheter was superior to PS and PS+CHX coated catheters in inhibiting the adherence of E. coli and S. epidermidis (FIGS. 4 and 6). However, PS+CHX combination-coated catheter showed an enhanced anti-adherence effect against P. aeruginosa (FIG. 5).

### Example 4

### Durability of Inhibitory Activity of Protamine Sulfate (PS) and Chlorhexidine Salt (CHX) Combination-Coated Silicone Catheter

The antimicrobial activity of PS+CHX coated 1 cm silicone catheter sections was assessed using Kirby-Bauer technique as previously described by Sheretz et al. (Antimicrob. Agents. Chemother., 33:1174-1178, 1989). The catheters were coated by dipping in a PS (100 mg/ml)+CHX (400 mg/ml) solution followed by drying as described by in U.S. Pat. No. 6,475,434. The catheter sections were gas-sterilized with ethylene oxide. Catheter-associated microorganisms such as E. coli. Proteus mirabilis, Pseudomonas aeruginosa, Klebsiella pneumoniae, Enterococcus faecalis, Vancomycin Resistant Enterococci (VRE), Staphylococcus epidermidis, Staphylococcus aureus, and Candida albicans were grown in nutrient broth for 18 hours at 37° C. An appropriate inoculum of each bacterial strain was used to prepare spread plates. The coated catheter sections were then carefully pressed into the center of each of the plates. Following incubation for 24 hours at 37° C., the zones of inhibition surrounding each of the sections were measured at the aspects of perpendicular to the long axes. After measuring the zones of inhibition, the sections were transferred onto fresh spread plates inoculated with respective test organism and incubated for 24 hours at 37° C. again. The zones of inhibition surrounding each of the sections were measured again. This procedure was repeated for determining the durability of inhibitory activity of coated catheter sections for 3 days, 7 days and 10 days with each test organism. The inhibitory activity of coated catheter sections against Klebsiella pneumoniae, VRE, and Pseudomonas aeruginosa lasted for only 3 days (Table 2). However, the coated catheter sections showed a significant inhibitory activity against E. coli, Staphylococcus epidermidis, Staphylococcus aureus, and Candida albicans even after 10 days of passage.

TABLE 2
Durability of inhibitory activity of the protamine sulfate (PS) +
chlorhexidine salt (CHX)-coated silicone catheter segments

| | Inhibition Zone (mm) | | | | |
|---|---|---|---|---|---|
| Organism | Day 0 | Day 1 | Day 3 | Day 7 | Day 10 |
| E. coli | 14 | 10 | 11 | 10 | 8 |
| Proteus mirabilis | 8 | 0 | 0 | 0 | 0 |
| Pseudomonas aeruginosa | 6 | 6 | 11 | 0 | 0 |
| Klebsiella pneumoniae | 10 | 6 | 6 | 0 | 8 |
| Enterococcus faecalis | 13 | 8 | 9 | 6 | 6 |
| Vancomycin Resistant | 13 | 9 | 9 | 7 | 0 |
| Enterococci (VRE) | | | | | |
| Staphylococcus | 19 | 16 | 13 | 15 | 12 |
| epidermidis | | | | | |
| Staphylococcus aureus | 21 | 13 | 14 | 8 | 10 |
| Candida albicans | 17 | 13 | 8 | 8 | 8 |

### Example 5

### Durability of Anti-Adherence Activity of Protamine Sulfate (PS) and Chlorhexidine Salt (CHX) Combination-Coated Silicone Catheter

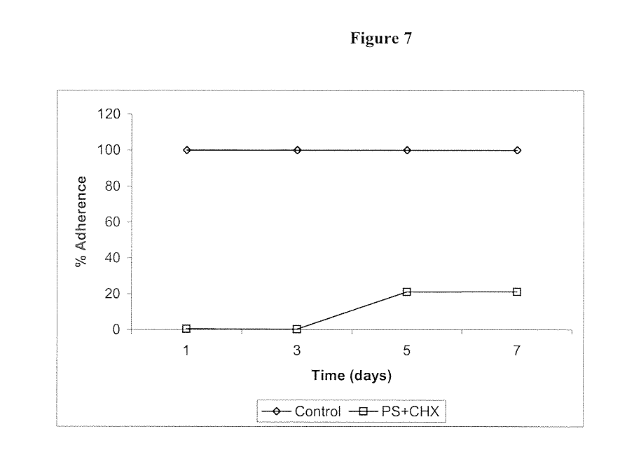

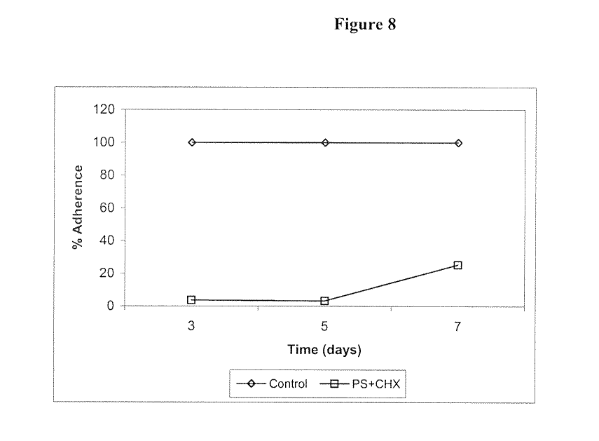

The ability of PS+CHX coated silicone catheters to resist bacterial colonization for a period of 7 days was tested by exposing uncoated and coated sections (in duplicate) to E. coli and Staphylococcus epidermidis. The silicone catheters were coated with PS (100 mg/ml)+CHX (400 mg/ml), and gas-sterilized with ethylene oxide. The coated and uncoated catheter sections were incubated in sterile artificial urine at 37° C. separately for 7 days at 100 rpm prior to challenging with the bacteria. Artificial urine in the flask was replaced with fresh artificial urine every 24 hours. Both coated and uncoated catheter segments (in triplicate) were removed at time intervals of 1, 3, 5, and 7 days and gently rinsed with sterile water. Further, they were challenged with the above test organisms one at a time. Following the incubation, the catheter sections were rinsed 3 times gently with sterile water and incubated in a test organism's culture broth at 37° C. for 3 hours at 100 rpm. After 3 hours of incubation, the sections were washed twice gently. Each washed segment was transferred into a sterile tube containing 1 ml sterile water and subjected to sonication for 30 seconds followed by 1 minute vortexing. Further, each section was serially diluted using sterile water and plated on LB agar. The plates were incubated for 24 hours at 37° C. and the colonies forming units (CFU) were counted. This procedure was repeated for each time interval. The PS+CHX coated catheter sections were effective in preventing bacterial cells adhering, as about 80% inhibition of adherence of both bacterial strains at day 7 was observed (FIGS. 7-8).

### Example 6

### In Vivo Efficacy of Protamine Sulfate (PS) and Chlorhexidine Salt (CHX) Combination-Coated Silicone Catheter

An in vivo efficacy study was conducted using a previously repotted rabbit model with slight modifications (Darouiche, et al., J. Heart. Valve. Dis., 11:99-104, 2002). This preliminary study was to assess the in vivo efficacy of silicone catheter coated with PS (100 mg/ml)+CHX (400 mg/ml) in preventing E. coli infection of subcutaneously implanted segments of silicone catheters. The silicone catheters were coated with PS (100 mg/ml)+CHX (400 mg/ml), and gas-sterilized with ethylene oxide. A total of 15 uncoated 1-cm segments of silicone catheters and 15 coated catheter segments were implanted subcutaneously in the back of a total of 4 rabbits that had received a single dose of vancomycin (20 mg/kg body weight) for prophylaxis against gram-positive skin microflora. Each device was inoculated with 50 μl of 2×10⁴ CFU/ml of clinical isolate of E. coli and wounds were then closed. 2 mg/kg body weight of ketoprofen was injected into each rabbit intramuscularly (IM) daily as an anti-inflammatory/analgesic. After 7 days, the four rabbits were sacrificed. The devices were explanted and cultured by using the sonication technique and plating. Swab cultures were obtained from surrounding fluid collections. Although 3 out of 15 (20%) uncoated segments were colonized by E. coli, all 15-coated segments were completely flee from bacterial colonization (Table 3).

TABLE 3
In vivo efficacy of protamine sulfate (PS) and chlorhexidine salt
(CHX)-coated silicone catheter

| Test | No. of | No. of Segments | % Infection |
|---|---|---|---|
| Group | Rabbits | Implanted | (after 7 days) |
| Control | 1 | 4 uncoated | 20 |
| | 2 | 4 uncoated | |
| | 3 | 4 uncoated | |
| | 4 | 3 uncoated | |
| Experimental | 1 | 4 coated | 0 |
| | 2 | 4 coated | |
| | 3 | 4 coated | |
| | 4 | 3 coated | |

### Example 7

### In Vivo Efficacy of Silicone Bladder Catheters Coated with Chlorhexidine+Protamine

The objectives of this Example were to: (1) confirm the in vivo efficacy of catheters coated with chlorhexidine/protamine as compared with uncoated catheters, (2) to compare the rates of device colonization and device-related infections by E. coli for catheters coated with chlorhexidine/protamine vs. catheters coated with hydrogel-silver, (3) to show that catheters coated with chlorhexidine/protamine were useful for preventing growth or proliferation of biofilm embedded microorganisms and, and (4) to show that catheters coated with chlorhexidine/protamine were useful in protecting against device-related infection.

An animal study was done using an established model of E. coli infection of medical devices inserted subcutaneously in the back of rabbits. Female New Zealand white, specific pathogen-free rabbits (body weight 2-3 kg) were anesthetized by receiving intramuscular injection (0.5 ml/kg body weight) of a mixture of ketamine (70 mg/kg body weight) and acepromazine (2 mg/kg body weight). To simulate the practice of administering perioperative antibiotic prophylaxis in human patients, each animal received immediately after induction of anesthesia an intramuscular (IM) injection of vancomycin (20 mg/kg) that was active against gram-positive organisms but not against E. coli. The backs of rabbits were shaved, then prepared and draped in a sterile fashion. Six (2 chlorhexidine/protamine-coated, 2 hydrogel-silver-coated, and 2 uncoated) 2-cm long catheter segments were subcutaneously inserted 3-4 cm lateral to the spine and away from each other. A total of 84 devices were placed in 14 rabbits. 10⁵ CFU of pathogenic of E. coli strain 2131 (a clinical isolate from a patient with catheter-related UTI) was inoculated onto the surface of inserted device and wounds were sutured. Rabbits were monitored daily for signs of local infection, sepsis, or major distress. Rabbits were sacrificed at 1 week and the following studies were done:

a. Quantitative cultures from devices by using the sonication technique, and

b. Qualitative swab culture of the site adjacent to the device.

The two primary outcomes of the study were device colonization (defined as growth of E. coli from quantitative sonication culture; detectability limit, 10 CFU) and device-related infection (defined as device colonization plus growth of E. coli from qualitative swab culture of the site surrounding the device). The rates of device colonization and device-related infection were compared between the different groups by using a 2-tailed Fisher's exact test with 90% power. A P value of ≦0.05 indicated significant differences.

The secondary outcome of the mean bacterial CFU retrieved from removed catheters was compared between the three groups by using the two-sample T test with unequal variance. A P value of ≦0.05 indicated significant differences.

Two of 28 (7%) chlorhexidine/protamine-coated catheters, 25 of 28 (89%) silver/hydrogel-coated catheters, and 18 of 28 (64%) uncoated catheters became colonized with E. coli. The chlorhexidine/protamine-coated catheters were significantly less likely to be colonized than either silver/hydrogel-coated catheters (P<0.001) or uncoated catheters (P=0.0016). There was no significant difference (P=0.51) in the rate of colonization of silver/hydrogel-coated vs. uncoated catheters.

One of 28 (4%) chlorhexidine/protamine-coated catheters. 12 of 28 (43%) silver/hydrogel-coated catheters, and 14 of 28 (50%) uncoated catheters developed device-related infection due to E. coli. The chlorhexidine/protamine-coated catheters were significantly less likely to cause device-related infection than either silver/hydrogel-coated catheters (P=0.046) or uncoated catheters (P=0.013). There was no significant difference (P=1.69) in the rate of device-related infection between the silver/hydrogel-coated vs. uncoated catheters.

The mean number of CFU was 4.6×10⁵ in the chlorhexidine/protamine group, 2.5×10⁶ in the silver-hydrogel group, and 8.3×10⁶ in the uncoated group. The mean number of CFU was significantly lower (P=0.031) on the surfaces of chlorhexidine/protamine-coated catheters than uncoated catheters. There were no significant differences in the mean number of cfu when comparing silver/hydrogel-coated catheters with either chlorhexidine/protamine-coated catheters (P=0.22) or uncoated catheters (P=0.13).

These results (Table 4) show that coating of catheters with chlorhexidine/protamine but not with silver/hydrogel protects against device colonization and device-related infection. The minimum detectability for device cultures was 10 CFU per device. 50 μl of 2×10⁶ CFU/ml or 1×10⁵ CFU of absolute inoculum was used. 2 mg/kg of ketoprofen was injected in each rabbit IM daily as an anti-inflammatory/analgesic. 20 mg/kg of vancomycin was given pre-operatively as a prophylactic antibiotic. External diameter of the silicone urinary catheter was 4 mm. 2 cm segments of uncoated catheters were used. The cultures from the blood drawn prior to sacrificing rabbits were all negative.

TABLE 4
In-vivo Activity of Antimicrobial Coated Urinary Silicone Catheters
against E. coli strain 2131.

| | No. of days | | Device culture | Site swab |
|---|---|---|---|---|
| Device | implanted | Device treatment | (total CFU) | (total CFU) |
| 5-3 | 7 | PA/CH | 0 | − |
| 5-4 | 7 | PA/CH | 0 | − |
| 6-1 | 7 | PA/CH | 0 | − |
| 6-6 | 7 | PA/CH | 0 | − |
| 7-2 | 7 | PA/CH | 0 | − |
| 7-5 | 7 | PA/CH | 0 | − |
| 8-2 | 7 | PA/CH | 0 | − |
| 8-5 | 7 | PA/CH | 0 | − |
| 9-3 | 7 | PA/CH | 0 | − |
| 9-4 | 7 | PA/CH | 0 | − |
| 10-1 | 7 | PA/CH | 0 | − |
| 10-6 | 7 | PA/CH | 0 | − |
| 11-2 | 7 | PA/CH | 1.7 × 10² | − |
| 11-5 | 7 | PA/CH | 0 | − |
| 12-3 | 7 | PA/CH | 0 | − |
| 12-4 | 7 | PA/CH | 0 | − |
| 13-1 | 7 | PA/CH | 0 | − |
| 13-6 | 7 | PA/CH | 0 | − |
| 14-2 | 7 | PA/CH | 0 | − |
| 14-5 | 7 | PA/CH | 0 | − |
| 15-3 | 7 | PA/CH | 0 | − |
| 15-4 | 7 | PA/CH | 0 | + |
| 16-2 | 7 | PA/CH | 0 | − |
| 16-5 | 7 | PA/CH | 0 | − |
| 17-3 | 7 | PA/CH | 1.3 × 10⁷ | + |
| 17-4 | 7 | PA/CH | 0 | − |
| 18-1 | 7 | PA/CH | 0 | − |
| 18-6 | 7 | PA/CH | 0 | − |
| 5-2 | 7 | Ag | 4.2 × 10⁶ | + |
| 5-5 | 7 | Ag | 2.4 × 10⁶ | + |
| 6-3 | 7 | Ag | 7.7 × 10² | + |
| 6-4 | 7 | Ag | 8.2 × 10⁴ | + |
| 7-3 | 7 | Ag | 7.0 × 10¹ | − |
| 7-4 | 7 | Ag | 4.4 × 10⁷ | + |
| 8-1 | 7 | Ag | 3.6 × 10³ | − |
| 8-6 | 7 | Ag | 1.4 × 10⁶ | + |
| 9-1 | 7 | Ag | 2.0 × 10⁶ | + |
| 9-6 | 7 | Ag | 3.8 × 10⁶ | − |
| 10-2 | 7 | Ag | 1.9 × 10⁵ | − |
| 10-5 | 7 | Ag | 0 | − |
| 11-3 | 7 | Ag | 5.6 × 10⁶ | − |
| 11-4 | 7 | Ag | 1.0 × 10⁴ | − |
| 12-2 | 7 | Ag | 4.2 × 10² | − |
| 12-5 | 7 | Ag | 1.4 × 10² | − |
| 13-2 | 7 | Ag | 6.8 × 10⁵ | + |
| 13-5 | 7 | Ag | 1.8 × 10⁶ | + |
| 14-3 | 7 | Ag | 0 | − |
| 14-4 | 7 | Ag | 2.5 × 10³ | − |
| 15-1 | 7 | Ag | 4.3 × 10³ | − |
| 15-6 | 7 | Ag | 3.1 × 10⁴ | − |
| 16-1 | 7 | Ag | 2.5 × 10⁵ | + |
| 16-6 | 7 | Ag | 1.1 × 10⁶ | + |
| 17-2 | 7 | Ag | 1.8 × 10⁶ | + |
| 17-5 | 7 | Ag | 1.8 × 10⁵ | − |
| 18-3 | 7 | Ag | 0 | − |
| 18-4 | 7 | Ag | 1.3 × 10³ | − |
| 5-1 | 7 | Uncoated | 2.4 × 10⁷ | + |
| 5-6 | 7 | Uncoated | 3.6 × 10⁷ | + |
| 6-2 | 7 | Uncoated | 1.5 × 10³ | + |
| 6-5 | 7 | Uncoated | 6.4 × 10⁴ | + |
| 7-1 | 7 | Uncoated | 5.6 × 10⁵ | + |
| 7-6 | 7 | Uncoated | 7.8 × 10⁷ | + |
| 8-3 | 7 | Uncoated | 0 | − |
| 8-4 | 7 | Uncoated | 0 | − |
| 9-2 | 7 | Uncoated | 1.6 × 10⁵ | − |
| 9-5 | 7 | Uncoated | 1.6 × 10⁴ | + |
| 10-3 | 7 | Uncoated | 4.0 × 10¹ | − |
| 10-4 | 7 | Uncoated | 0 | − |
| 11-1 | 7 | Uncoated | 7.6 × 10⁶ | + |
| 11-6 | 7 | Uncoated | 4.2 × 10⁷ | + |
| 12-1 | 7 | Uncoated | 0 | − |
| 12-6 | 7 | Uncoated | 2.3 × 10⁷ | + |
| 13-3 | 7 | Uncoated | 0 | − |
| 13-4 | 7 | Uncoated | 0 | − |
| 14-1 | 7 | Uncoated | 2.5 × 10⁵ | + |
| 14-6 | 7 | Uncoated | 0 | − |
| 15-2 | 7 | Uncoated | 0 | − |
| 15-5 | 7 | Uncoated | 1.0 × 10² | + |
| 16-3 | 7 | Uncoated | 6.7 × 10² | − |
| 16-4 | 7 | Uncoated | 3.0 × 10 | − |
| 17-1 | 7 | Uncoated | 2.0 × 10⁷ | + |
| 17-6 | 7 | Uncoated | 7.0 × 10⁵ | + |
| 18-2 | 7 | Uncoated | 0 | − |
| 18-5 | 7 | Uncoated | 0 | − |

### Example 8

### Minimum Inhibitory Concentrations of Chlorhexidine (CHX), Protamine Sulfate (PS), and CHX and PS Combination for Bacteria Associated with Biofilms in Industries

E. coli, K. pneumoniae, P. aeruginosa, S. aureus, B. cereus, S. thermophilus, L. monocytogenes and C. perfringens are bacteria frequently encountered in a wide variety of industries, including dairy, pulp and paper mills, food and beverage manufacturing industry, water treatment facilities, etc. Some of them are commonly found in a variety of consumer products and household items, and are often found in, for example, kitchens, bathrooms, HVAC systems, humidifiers, vacuum cleaners, toys and the like.

The minimum inhibitory concentrations (MICs) of chlorhexidine (CHX) and protamine sulfate (PS) alone and CHX and PS combination for E. coli, Klebsiella pneumoniae, Pseudomonas aeruginosa, Staphylococcus aureus, Bacillus cereus, Streptococcus thermophilus, Listeria monocytogenes and Clostridium perfringens are determined using a broth microdilution assay in 96-well microtiter plate as described previously (Amsterdam, D. 1996, In: V. Loman, Ed., “Antibiotics in laboratory medicine”, p. 52-111, Williams and Wilkins, Baltimore, Md.). Briefly, bacterial strains were grown overnight at 37° C. with 100 rpm shaking in TSB and diluted to approximately 10⁵ CFU/ml. Antimicrobials CHX (50 to 0.098 to μg/ml) and PS (200 to 0.195 μg/ml) alone and together were serially diluted in TSB (100 μl), and 100 μl of bacterial suspension were added to each well. Plates were incubated at 37° C. for 24 h and were read at 600 nm using a microtiter plate reader (Multiskan Ascent, Labsystems, Helsinki, Finland). The MIC was taken to be the lowest concentration of antimicrobial that completely inhibits growth. The MIC for the combination of CHX and PS was found to be significantly lower than the MIC for either of CHX and PS alone. The combination of CHX and PS demonstrated enhanced inhibition of E. coli, K. pneumoniae and S. aureus growth (Table 5).

TABLE 5
MIC of chlorhexidine (CHX), protamine sulfate (PS) and CHX
and PS combination for bacteria associated with biofilms in industries

MIC (μg/ml)

| Organism | CHX | PS | CHX + PS |
|---|---|---|---|
| E. coli | 0.39 | >100 | 0.195 + 0.39* |
| K. pneumoniae | 6.25 | >200 | 3.125 + 12.5* |
| P. aeruginosa | 12.5 | 200 | 12.5 + 50 |
| S. aureus | 0.781 | 200 | 0.39 + 1.56* |
| B. cereus | 3.125 | >200 | 3.125 + 12.5 |
| S. thermophilus | <0.195 | 200 | 0.78 +< 0.195 |
| L. monocytogens | 3.125 | 200 | 3.125 + 12.5 |
| C. perfringens | 1.56 | >200 | 1.56 + 6.25 |

*Combination showing enhanced inhibition.

### Example 9

### Minimum Inhibitory Concentrations of Chlorhexidine (CHX), Protamine Sulfate (PS), and CHX and PS Combination for Oral Bacteria Associated with Plaque, Caries and Periodontal Diseases

S. mutans and S. sobrinus are the major oral bacteria associated with dental caries. They are the primary colonizers of teeth resulting in the early dental plaque formation. Other oral bacteria such as A. naeslundii, F. nucleatum, P. gingivalis and P. intermedia are associated with dental plaque and periodontal diseases.

The minimum inhibitory concentrations (MICs) of chlorhexidine (CHX) and protamine sulfate (PS) alone and CHX and PS combination for S. mutans, S. sobrinus and A. naeslundii were determined using a broth microdilution assay in 96-well microtiter plate as described previously (Amsterdam, D. 1996, In: V. Loman, Ed., “Antibiotics in laboratory medicine”, p. 52-111, Williams and Wilkins, Baltimore, Md.). S. mutans and S. sobrinus were grown overnight at 37° C. with 100 rpm shaking in THYE broth supplemented with 0.01% hog gastric mucin, and A. naeslundii was grown in TSB-YK broth, and diluted to approximately 10⁵ CFU/ml. Antimicrobials CHX (50 to 0.098 to μg/ml) and PS (200 to 0.195 μg/ml) alone and together were serially diluted in THYE (100 μl), and 100 μl of bacterial suspension is added to each well. Plates were incubated at 37° C. for 24 h and were read at 600 nm using a microtiter plate reader (Labsystems, Multiskan Ascent, Helsinki, Finland). The MIC was taken to be the lowest concentration of antimicrobial that completely inhibits growth. The MIC for the combination of CHX and PS was found to be significantly lower than the MIC for either of CHX and PS alone, and the combination of CHX and PS is found to be synergistic in the inhibition of microbial growth for Streptococcus spp tested (Table 6).

TABLE 6
Minimum inhibitory concentrations of chlorhexidine (CHX), protamine
sulfate (PS), and CHX and PS combination for oral bacteria
associated with plaque, caries and periodontal diseases.

| | CHX | PS | |
|---|---|---|---|
| Bacterial Strain | (μg/ml) | (μg/ml) | CHX + PS (μg/ml) |
| S. mutans UA 159 | 6.25 | >200 | 0.78 + 3.12 |
| S. sobrinus HNG | 1.56 | >200 | 1.56 + 6.25 |
| 909S | | | |
| A. naeslundii ATCC | 50 | >200 | 12.5 + 50 |
| 12104 | | | |

### Example 10

### Enhancing Effect of Protamine Sulfate (PS) on the Activity of Chlorhexidine (CHX) Against Biofilm-Embedded Bacteria Associated with Biofilms in Industries

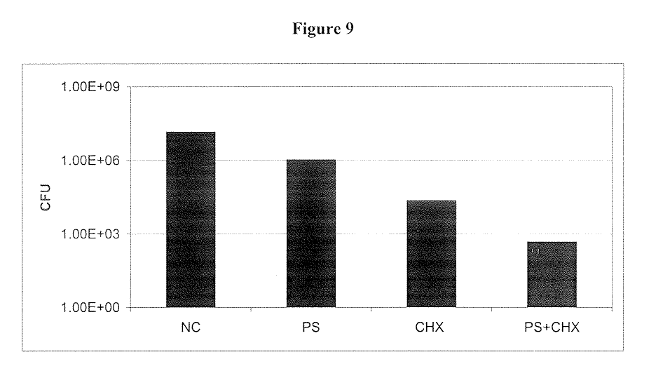

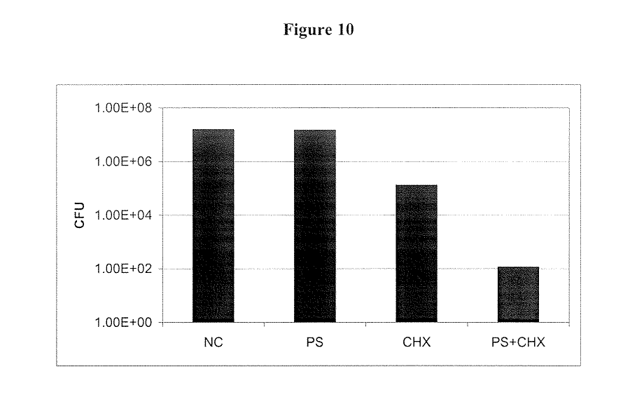

Biofilms were assayed using a modified quantitative biofilm assay method as described previously (Jackson, D. W. et al., J. Bacteriol. 184: 290-301, 2002). The overnight cultures of E. coli and B. cereus were diluted to 5% in TSB. Biofilms of bacteria were grown at 37° C. in 12-well tissue culture polystyrene plates (Corning Inc., New York). Aqueous solutions of CHX and PS were prepared separately and appropriate volume of each were added to 12-well plates individually and in combinations. The total volume of each well was made up to 2 ml with sterile distilled water. The wells without antimicrobials served as control. After 24 h incubation, the media containing planktonic cells in each well were removed, and biofilm was rinsed with PBS. After adding 2 ml of PBS to each well, the plate was sonicated for 15 seconds, and dislodged biofilm was mixed well with the pipette tip. Further, the 1 ml suspension from each well was serially diluted (10-fold dilution) and plated 100 μl of each dilution on TSA. The plates are incubated at 37° C. for 24 h and colonies were counted. Plates from the wells treated with CHX and PS in combination contain significantly fewer colonies than either treatment on its own. A significantly lower concentration of CHX and PS was required for an equivalent number of colonies formed, as compared to CHX or PS treatment alone. The combination of CHX and PS demonstrated enhanced inhibition of the growth of biofilm embedded E. coli and B. cereus (FIGS. 9 and 10).

What is claimed is:

1. A composition for decreasing growth or proliferation of biofilm embedded microorganisms, said composition comprising: (a) a cationic polypeptide and (b) a bis-guanide or a salt thereof.

2. An oral care consumable product comprising the composition of claim 1.

3. The oral care consumable product according to claim 2, wherein said oral care consumable is selected from the group consisting of a toothpaste, a mouth wash, a dental floss, a chewing gum and a breath mint.

4. A cleaning product comprising the composition of claim 1.

5. The cleaning product of claim 4, wherein said cleaning product is selected from the group consisting of a general household disinfectant, a window cleaner, a bathroom cleaner, a kitchen cleaner, a floor cleaner, a laundry detergent, a cleaning supply; a fruit and vegetable wash; and a fabric softener.

6. A cosmetic product comprising the composition of claim 1.

7. The cosmetic product according to claim 6, wherein said cosmetic product is selected from a group consisting of: face powder, a lip balm, a lipstick, an eyeliner, and a mascara.

8. A plastic product comprising the composition of claim 1.

9. The plastic product of claim 8, wherein said plastic product is selected from the group consisting of: a toy; a washing machine; a toothbrush; a denture; a mouth guard; a dairy line; a dairy line filter; a water line; a line used in food and beverage manufacturing; a cosmetic container; a bottle; a vacuum cleaner; a vacuum cleaner filter; an oil or gas pipe; a window frame; a door; a door frame; a humidifier; a humidifier filter; an outdoor pond liner; an air filter; a toilet seat; a tupperware; a shower; a shower head; a vacuum cleaner bag; an HVAC system; an HVAC filter; a cooling tower; a sink; a tap and water spout; a water jug; a water sprinkling line; a water sprinkler; a bathtub; a garbage bag; a dishwasher; a bathroom tile; a bathroom fixture; a dish drying tray; a whirlpool bathtub; a toilet; a toilet lid; a fish pond; a swimming pool; a swimming pool liner; a swimming pool skimmer; a swimming pool filter; a planter; a hot tub line; a hot tub filter; a medical instrument; a dental instrument; a washing machine liner; an animal water dish; a dish washer liner; a food storage container; a beverage storage container; a dish; a plate; a bowl; a cup; a fork; a knife; a spoon; a utensil; a cutting board; a garden hose; a bird bath; a hot tub; and a counter top.

10. A wound care product comprising the composition of claim 1.

11. The wound care product of claim 10, wherein said wound care product is selected from the group consisting of band aids, non-resorbable gauze/sponge dressing, hydrophilic wound dressing, occlusive wound dressing, hydrogel wound and burn dressing, spray-applicator, ointments, lotions, cream and suture.

12. A product coated with the composition of claim 1.

13. The product of claim 12, wherein said product is selected from the group consisting of: a denture; a mouth guard; a dairy line; a water line; an adhesive bandage; a component of an HVAC system; a component of a water treatment facility; a component of a vacuum or a vacuum cleaner; a vacuum cleaner bag; a vacuum cleaner filter; an air filter; a component of a cooling tower; a toy; a window; a door; a window frame; a door frame; a medical instrument; a dental instrument; a bathroom tile; a kitchen tile; an animal water dish; a washing machine; a dish washer; a towel; a dish; a bowl; a utensil; a cup; a glass; a cutting board; a dish drying tray; a whirlpool bathtub; a sink; a toilet; a toilet seat; a swimming pool; a bird bath; a planter; a garden hose; a fish pond; an oil pipe; a gas pipe; a dairy line filter; a line used in food and beverage manufacturing; a cosmetic container; an outdoor pond liner; a tap and water spout; a humidifier; a humidifier filter; a bathroom tile; a bathroom fixture; a toilet lid; a swimming pool liner; a swimming pool skimmer; a swimming pool filter; a hot tub line; a hot tub filter; a washing machine liner; a dishwasher liner; an animal water dish; a food storage container; a beverage storage container; a plate; a cup; a fork; a knife; a spoon; a garbage bag; and a countertop.

14. A paint comprising the composition of claim 1.

15. A method of preparing an object comprising treating at least one surface of the object with the composition of claim 1.

16. The method of claim 15, wherein the object is selected from the group consisting of:
a toothbrush; dental floss; a denture, a mouth guard; a dairy line; a dairy line filter; a water line; a line used in food and beverage manufacturing; a cosmetic container; a plastic bottle; a vacuum; a vacuum cleaner; a tap and water spout; an outdoor pond liner; equipment involved in the leeching process or mining; a water jug; a water sprinkling line; a water sprinkler; a garbage bag; tupperware; a bath tub; a sink; a shower; a shower head; a dishwasher; a vacuum cleaner bag; a vacuum cleaner filter; an oil or gas pipe; a window frame; a door; a door frame; a humidifier; a humidifier filter; HVAC systems and filters thereto, a toy; a cooling tower; a medical instrument; a dental instrument; a washing machine; a washing machine liner; a dishwasher; a dishwasher liner; a dish; a plate; a cup; a utensil; a bowl; a fork; a knife; a spoon; an animal water dish; a bathroom tile; a bathroom fixture; a sealant; grout; a towel; a food storage container; a beverage storage container; a cutting board; a dish drying tray; a whirlpool bathtub; a toilet; a toilet lid; a toilet seat; a fish pond; a swimming pool; a swimming pool liner; a swimming pool skimmer; a swimming pool filter; a bird bath; a garden hose; a planter; a hot tub; a hot tub lines; a hot tub filter; and a counter top.

17. The oral care consumable product according to claim 2, wherein the cationic polypeptide is protamine sulfate and the bis-guanide is a chlorhexidine base or salt.

18. The cleaning product according to claim 4, wherein the cationic polypeptide is protamine sulfate and the bis-guanide is a chlorhexidine base or salt.

19. The plastic product according to claim 8, wherein the cationic polypeptide is protamine sulfate and the bis-guanide is a chlorhexidine base or salt.

20. The wound care product according to claim 10, wherein the cationic polypeptide is protamine sulfate and the bis-guanide is a chlorhexidine base or salt.
